(19) United States
(12) Patent Application Publication
(10) Pub. No.: US 2022/0082507 A1
(43) Pub. Date: Mar. 17, 2022

(54) PRODUCT SUBSTANCE INSPECTION SYSTEM AND PRODUCT SUBSTANCE INSPECTION METHOD

(71) Applicant: Quanta Computer Inc., Taoyuan City (TW)

(72) Inventors: Chen-Chung LEE, Taoyuan City (TW); Chia-Hung LIN, Taoyuan City (TW); Yao-Kun LEE, Taoyuan City (TW); Yu-Ting TSENG, Taoyuan City (TW)

(21) Appl. No.: 16/951,007

(22) Filed: Nov. 18, 2020

(30) Foreign Application Priority Data
Sep. 14, 2020 (TW) 109131433

(51) Int. Cl. G01N 21/88

(52) U.S. Cl. CPC G01N 21/8851; G01N 2021/936

(57) ABSTRACT

A product substance inspection system includes an expansion module, an inspection module, and a preference-setting module. The expansion module expends regulatory information into a plurality of substance thresholds. The inspection module obtains material composition corresponding to a constituent from the aggregate material composition stored in a database, and compares each element in the material composition with the corresponding substance threshold. When the inspection module determines that the value of a specific element in the material composition exceeds the corresponding substance threshold, the preference-setting module replaces the constituent to which the specific element belongs according to the preference settings.

### CROSS REFERENCE TO RELATED APPLICATIONS

This application claims priority of Taiwan Patent Application No. 109131433, filed on Sep. 14, 2020, the entirety of which is incorporated by reference herein.

### BACKGROUND OF THE INVENTION

### Field of the Invention

The present disclosure relates to an inspection system and, in particular, to a product substance inspection system and a product substance inspection method suitable for product substances.

### Description of the Related Art

With the rise of global awareness of environmental protection, countries have begun to formulate relevant laws and regulations on environmental issues. At the same time, the impact of chemical substance restrictions on the global management of supply chains is increasing. At present, most companies are dealing with chemical-substance management, nothing more than collecting self-declarations from suppliers or doing part of the whole-machine or product testing, which often causes great risks in chemical-substance management.

Due to the increasing number of items of concern about hazardous substances and restricted substances in some laws and regulations, the collection of self-declarations from suppliers or product testing takes a lot of time and human resources. However, effective management and data reuse cannot be achieved in the management of chemical supplies.

Therefore, how to detect whether each substance in a product is in compliance with laws and regulations, and to provide an improvement suggestion mechanism to ensure the effective management and use of supply-chain materials, has become one of the problems that need to be resolved in the field.

### BRIEF SUMMARY OF THE INVENTION

In accordance with one feature of the present invention, the present disclosure provides a product substance inspection system. The product substance inspection system includes an expansion module, an inspection module and a preference-setting module. The expansion module expends regulatory information into a plurality of substance thresholds. The inspection module obtains material composition corresponding to a constituent from the aggregate material composition stored in a database, and compares each element in the material composition with the corresponding substance threshold. When the inspection module determines that the value of a specific element in the material composition exceeds the corresponding substance threshold, the preference-setting module replaces the constituent to which the specific element belongs according to the preference settings.

In accordance with one feature of the present invention, the present disclosure provides a product substance inspection method. The product substance inspection method includes the following steps: expending regulatory information into a plurality of substance thresholds by an expansion module; obtaining the material composition of a constituent from the aggregate material composition stored in a database by an inspection module; and comparing each element in the material composition with the corresponding substance threshold by a preference-setting module. When the inspection module determines that the value of a specific element in the material composition exceeds the corresponding substance threshold, a preference-setting module replaces the constituent to which the specific element belongs according to the preference settings.

Therefore, the product substance inspection system and the product substance inspection method provide a way to obtain one or more element corresponding to each constituent from a large amount of constituents, and automatically compare whether these elements comply with various laws and regulations. This saves a lot of labor costs and inspection time. In addition, when the product substance inspection system and product substance inspection find that some constituents in the end product do not comply with the regulations, a variety of preference settings can be applied to automatically select the most suitable substitute constituent for the user. This can ensure effective control of materials in the supply chain. In addition, the product substance inspection system and product substance inspection method can expend the material (or constituent) of the end product according to the bill of materials. Moreover, the product substance inspection system performs a tandem inspection with the aggregate material composition provided by the supplier and the test report provided by a third-party testing organization. The expended materials can be checked for compliance with product substance content (or element content) of the relevant regulations. In addition, the product substance inspection system and product substance inspection method can also determine whether the content of the analyzed test report is consistent with the aggregate material composition, list the details for the non-conforming substance items, and provide alternative material composition for improvement suggestions. In this way, alternative solutions can be quickly found, and effective risk management can be performed on the provided materials to ensure the safety of product materials. In addition, the product substance inspection system and the product substance inspection method can also be adjusted according to preference settings to automatically output improvement suggestions.

### BRIEF DESCRIPTION OF THE DRAWINGS

In order to describe the manner in which the above-recited and other advantages and features of the disclosure can be obtained, a more particular description of the principles briefly described above will be rendered by reference to specific examples thereof which are illustrated in the appended drawings. Understanding that these drawings depict only example aspects of the disclosure and are not therefore to be considered to be limiting of its scope, the principles herein are described and explained with additional specificity and detail through the use of the accompanying drawings in which:

FIG. 1 is a schematic diagram of a product substance inspection system in accordance with one embodiment of the present disclosure.

FIG. 2 is a flowchart of a product substance inspection method in accordance with one embodiment of the present disclosure.

FIG. 3 is a schematic diagram of a product substance inspection method in accordance with one embodiment of the present disclosure.

FIG. 4 is a schematic diagram of a product substance inspection method in accordance with one embodiment of the present disclosure.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

The present invention will be described with respect to particular embodiments and with reference to certain drawings, but the invention is not limited thereto and is only limited by the claims. It will be further understood that the terms “comprises,” “comprising,” “includes” and/or “including,” when used herein, specify the presence of stated features, integers, steps, operations, elements, and/or elements, but do not preclude the presence or addition of one or more other features, integers, steps, operations, elements, elements, and/or groups thereof.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements.

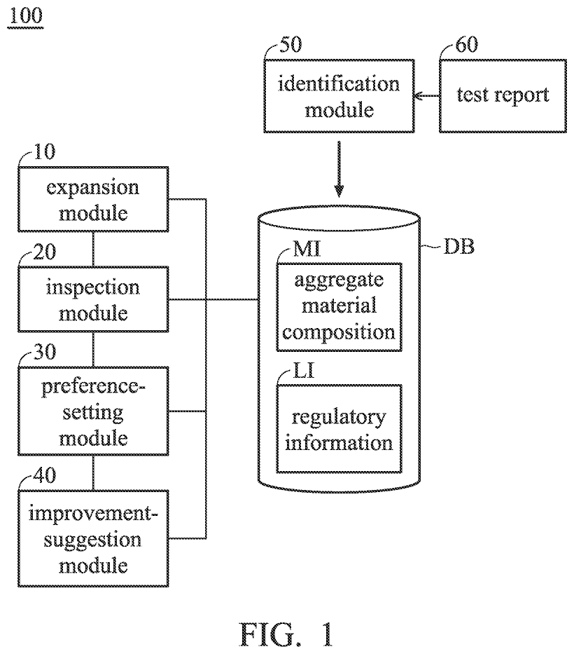

Refer to FIG. 1, FIG. 1 is a schematic diagram of a product substance inspection system 100 in accordance with one embodiment of the present disclosure. In one embodiment, as shown in FIG. 1, the product substance inspection system 100 includes an expansion module 10, a database DB, an inspection module 20 and a preference-setting module 30. In one embodiment, the product substance inspection system 100 further includes an improvement-suggestion module 40 and an identification module 50.

In one embodiment, the expansion module 10, the database DB, the inspection module 20, the preference-setting module 30, the improvement-suggestion module 40 and the identification module 50 can be implemented together or separately as, for example, a microcontroller, a microprocessor, a digital signal processor, an application specific integrated circuit (ASIC) or a logic circuit.

In one embodiment, the expansion module 10, the database DB, the inspection module 20, the preference-setting module 30, the improvement-suggestion module 40 and the identification module 50 can be implemented by software or firmware.

In one embodiment, the database DB can be stored in a storage device, and the storage device can be read-only memory, flash memory, floppy disk, hard disk, optical disk, flash drive, tape, or network accessible database or those familiar with the art can easily think of storage media with the same function to implement it.

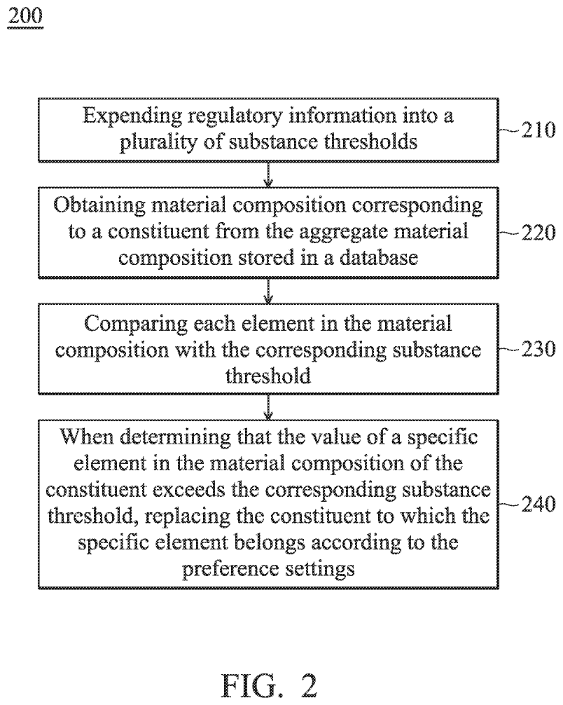

Please refer to FIGS. 1 and 2 together. FIG. 2 is a flowchart of a product substance inspection method 200 in accordance with one embodiment of the present disclosure. The product substance inspection method 200 can be implemented by the product substance inspection system 100 in FIG. 1.

In step 210, an expansion module 10 expends regulatory information LI into a plurality of substance thresholds.

In one embodiment, the expansion module 10 can receive the regulatory information LI selected or input from a user interface. In one embodiment, the regulatory information LI is, for example, but not limited to, the Restriction of Hazardous Substances (referred to as RoHS Regulations), EC Regulation No. 1907/2006, concerning Registration, Evaluation, Authorization and Restriction of chemicals (referred to as REACH regulations). REACH regulations list Substances of Very High Concern (SVHC), that is, hazardous substances.

In one embodiment, the expansion module 10 is responsible for expanding the regulations or preset regulations selected by the user to the material level as the input of the inspection module 20. For example, if the regulatory information LI obtained by the expansion module 10 is that the lead (Pb) content must not exceed 1,000 parts per million (ppm), then the expansion module 10 can expend the regulatory information LI into “Regulation 1: Lead content <1000 ppm”, the substance threshold of the substance representing the lead content is 1000 ppm, and the lead element in the constituent must be less than 1000 ppm to be considered legal.

In one embodiment, the expansion module 10 can use known word parsing algorithms, word segmentation algorithms, and keyword search algorithms to extract the substance thresholds of various elements from the regulatory information LI, for example, the substance thresholds of elements such as mercury (Hg) and cadmium (Cd), but not limited thereto.

In one embodiment, the expansion module 10 stores the expended regulatory information LI (including elements and their corresponding substance thresholds) into the database DB.

In one embodiment, the expansion module 10 can also expend the end product materials according to the bill of materials (BOM), and perform a tandem inspection with the aggregate material composition MI provided by the supplier and the test report 60 provided by a third-party testing organization. In one embodiment, each material corresponds to a constituent.

In step 220, the inspection module 10 obtains material composition corresponding to a constituent from the aggregate material composition MI stored in a database DB.

In one embodiment, the inspection module 20 stores the aggregate material composition MI in the database DB. In one embodiment, the aggregate material composition MI includes all the constituents (For example, the top cover of the laptop, the screen, the touchpad, and so on. Each is composed of at least one material) of an end product (for example, a laptop). Moreover, each of the constituents corresponds to its own material composition. The material composition contains at least one element of the constituent (for example, the top cover of the laptop contains elements such as lead, mercury, cadmium, etc.).

For example, the aggregate material composition MI can reflect that the top cover of a laptop includes 1200 ppm of lead, 200 ppm of mercury, and 80 ppm of cadmium, the screen of a laptop includes 1300 ppm of lead, 1100 ppm of mercury, and 30 ppm of cadmium, and the touchpad of a laptop includes 1100 ppm of lead and 200 ppm of mercury, as well as other information.

In other words, the constituent refers to a part or all of the various elements of the end product (for example, the top cover of the laptop, the screen, the touchpad, etc., each may use at least one constituent). Each constituent is composed of multiple elements (such as lead, mercury, and cadmium). This information (i.e., the aggregate material composition MI) can be obtained in advance by the supplier or a third-party testing organization and stored in the database DB. The third-party testing organization is, for example, an SGS certification organization.

In one embodiment, the identification module 50 receives a test report 60 of a end product from a third-party server (belonging to a third-party testing organization) or a vendor server (belonging to a supplier), and performs text analysis on the test report 60 (for example, using known string processing algorithms, segmentation algorithms, and keyword search algorithms). When the analyzed content of the test report 60 is consistent with the aggregate material composition MI, the aggregate material composition MI is stored in the database DB.

In one embodiment, when the parsed content of the test report 60 is inconsistent with the aggregate material composition MI, the parsed content of the test report 60 is stored in the database DB. Moreover, in the subsequent steps, regarding the reading of the aggregate material composition MI is replaced with the reading the content of analyzed test report 60.

In one embodiment, the identification module 50 can compare the aggregate material composition MI provided by the supplier with the aggregate material composition MI certified by a third-party testing organization to verify the correctness of the aggregate material composition MI. If the correctness of the aggregate material composition MI is correct, then the aggregate material composition MI is stored in the database DB.

In this way, it is possible to achieve the suitability assessment of the supplier's substance declaration content (the aggregate material composition MI) and the test report certified by a third-party testing organization (for example, test report 30) to ensure the contents of the substance (element or element) provided by the supply chain.

In step 230, the inspection module 20 compares each element in the material composition with the corresponding substance threshold.

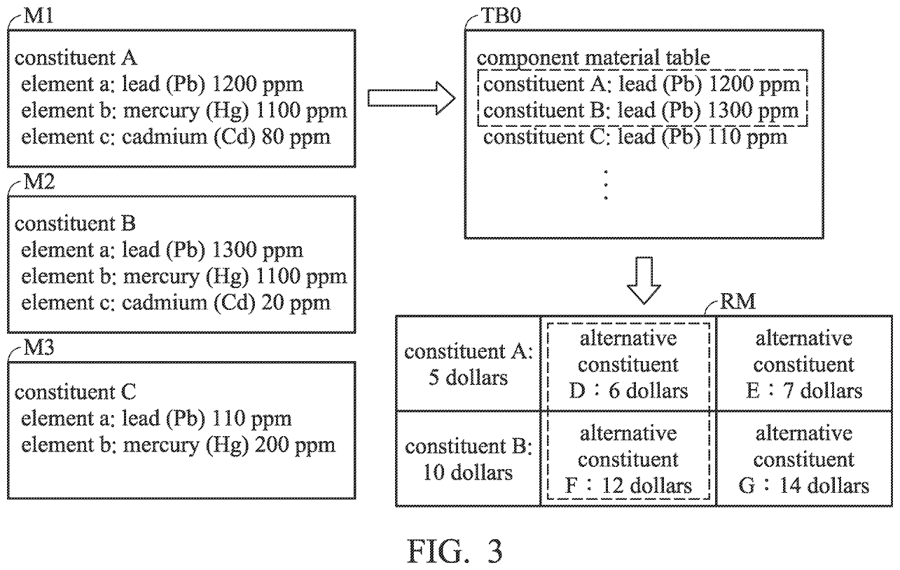

In an embodiment, please refer to FIG. 3, FIG. 3 is a schematic diagram of a product substance inspection method in accordance with one embodiment of the present disclosure. In FIG. 3, the inspection module 20 obtains the material composition M1 to M3 corresponding to the constituents A to C from the aggregate material composition MI stored in the database DB.

In this example, it can be seen from the constituent material composition M1: constituent A contains element a: lead (Pb) 1200 ppm, element b (Hg): mercury 200 ppm, element c: cadmium (Cd) 80 ppm. It can be seen from the material composition M2 that the constituent B contains element a: lead (Pb) 1300 ppm, element b: mercury (Hg) 1100 ppm, and element c: cadmium (Cd) 30 ppm. It can be seen from the constituent M3 that the constituent C contains element a: lead (Pb) 1100 ppm, mercury (Hg) 200 ppm.

In one embodiment, the expansion module 10 can also organize the material composition M1-M3 into a component material table TB0 and store it in the database DB.

In one embodiment, all material composition (for example, including material composition M1 to M3 and all other material composition constituting the laptop) of the end product (such as a laptop) can be regarded as the aggregate material composition MI.

In step 240, when the inspection module 20 determines that the value of a specific element in the material composition of the constituent exceeds the corresponding substance threshold, the preference-setting module 30 replaces the constituent to which the specific element belongs according to the preference settings.

In one embodiment, please refer to the cost table RM in FIG. 3, when the inspection module 20 determines that the value of a specific element in the material composition exceeds the corresponding substance threshold, the preference-setting module 30 selects an alternative constituent (for example, alternative constituent D) according to the preference setting to replace the constituent (for example, constituent A) to which the specific element belongs. The values of all the elements of the substitute constituent need to meet their corresponding substance thresholds.

In an embodiment, the specific element is, for example, lead, mercury, cadmium, or another element.

In one embodiment, please refer to FIG. 3, suppose that the expansion module 10 expends the regulatory information LI into “Law 1: Lead content <1000 ppm”, which means that the substance threshold for lead content is 1000 ppm. In other words, the lead elements in constituents A to C must be less than 1000 ppm to be considered legal. For example, it can be seen from the component material table TB0 that a certain component (for example, a touch panel) requires constituent A, constituent B, and constituent C to be composed. In the example in FIG. 3, the lead content of constituent A and constituent B (considered as one of the specific elements) is 1200 ppm and 1300 ppm respectively, both of which are higher than the lead content of 1000 ppm. Therefore, it is necessary to replace constituent A and constituent B with alternative constituents that comply with the regulatory information L1.

It can be seen from the cost table RM that the substitute constituent of constituent A (cost 5 dollars) is the substitute constituent D (cost 6 dollars) or the substitute constituent E (cost 7 dollars). Moreover, the substitute material of constituent B (cost 10 dollars) is the substitute constituent F (cost 12 dollars) or the substitute constituent G (cost 14 dollars). If the preference setting (which can be set in advance or the product substance inspection system 100 automatically sets according to a preference rule) is based on cost considerations, the preference-setting module 30 selects the alternative constituent D (the cost is lower than the alternative constituent E) according to the preference setting to replace constituent A, and selects alternative constituent F (the cost is lower than the alternative constituent G) to replace constituent B. The preference rule is, for example, the weight of the substitute constituent or the preset order of the substitute constituent.

However, the present applicant is not limited to thereto. The inspection module 20 can inspect the element content of all the constituents required by the entire end product or at least one element in the above-mentioned manner. When the inspection module 20 determines that the value of a specific element in the content of these elements exceeds the corresponding substance threshold (which may indicate that the content of harmful substances is too high), the preference-setting module 30 automatically selects the replacement constituent according to the preference setting (this substitute constituent is a material that complies with regulations) to replace the constituent to which this specific element belongs.

In one embodiment, the constituents (for example, constituent A and constituent B) that do not comply with the regulatory information L1 in the component material table TB0 can be selected and displayed on a display.

In one embodiment, the improvement-suggestion module 40 obtains a plurality of weights in the preference settings, and calculates weighted scores corresponding to each of a plurality of candidate substitute constituents according to these weights (for example, the candidate substitute constituent of constituent A is the substitute constituent D and the substitute constituent E), and the largest value among these weighted scores is selected as the substitute constituent (for example, substitute constituent E).

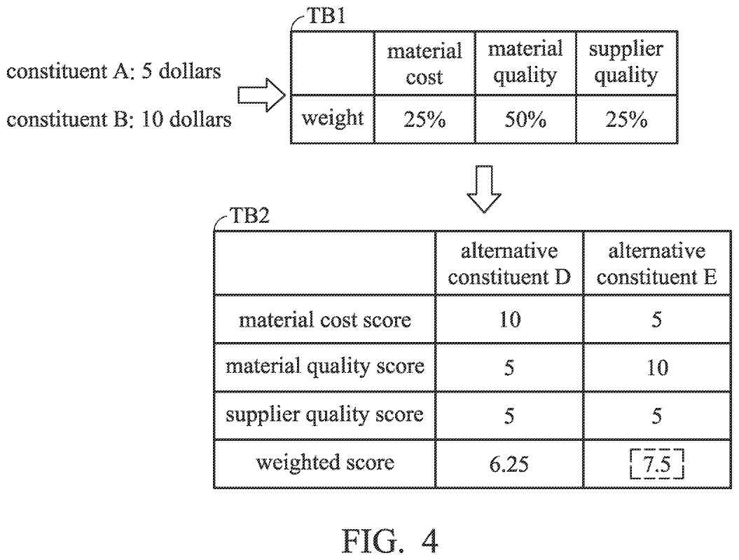

In an embodiment, please refer to FIGS. 3 to 4, and FIG. 4 is a schematic diagram of a product substance inspection method in accordance with one embodiment of the present disclosure. In this example, the preference setting includes material cost, material quality, and supplier quality. However, this is an example, and the preference setting items are not limited to thereto.

In one embodiment, the improvement-suggestion module 40 can receive the weight of each preference setting through an input interface (such as a keyboard, a touch screen, a mouse, a communication interface, etc.). In one embodiment, the weight of the preference setting is a preset value.

In FIG. 4, the weight of material cost is 25%, the weight of material quality is 50%, and the weight of supplier quality is 25%. From the foregoing and FIG. 3, it can be seen that taking constituent A as an example, the lead content of constituent A is higher than the lead content specified in the regulatory information L1 by 1000 ppm. Therefore, it is necessary to replace constituent A with a substitute constituent that complies with the regulatory information L1. From the weighting table TB1 and the score table TB2 in FIG. 4, the weighted score of the alternative constituent D and the weighted score of the alternative constituent E of constituent A can be calculated. For example, if the material cost score of substitute constituent D is 10, the material quality score is 5, and the supplier quality score is 5 (the scores can be defined in advance), then 10*25%+5*50%+5*25% gets a weighted score of 6.25. The material cost score of the alternative constituent E is 5, the material quality score is 10, and the supplier quality score is 5 (the scores can be defined in advance), then 5*25%+10*50%+5*25% gets a weighted score of 7.5. The improvement-suggestion module 40 selects the alternative constituent E corresponding to a higher weighted score to replace the constituent A.

Therefore, the user can select the preference setting and adjust the weight of each preference settings, so that the improvement-suggestion module 40 automatically selects the most suitable replacement constituent for the user.

In one implementation, the improvement-suggestion module 40 automatically checks whether the selected substitute constituent that is most suitable for the user complies with other regulations, and if it does not comply with other regulations, one of other substitute constituent is selected.

Therefore, the product substance inspection system and the product substance inspection method provide a way to obtain one or more element corresponding to each constituent from a large amount of constituents, and automatically compare whether these elements comply with various laws and regulations. This saves a lot of labor costs and inspection time. In addition, when the product substance inspection system and product substance inspection find that some constituents in the end product do not comply with the regulations, a variety of preference settings can be applied to automatically select the most suitable substitute constituent for the user. This can ensure effective control of materials in the supply chain. In addition, the product substance inspection system and product substance inspection method can expend the material (or constituent) of the end product according to the bill of materials. Moreover, the product substance inspection system performs a tandem inspection with the aggregate material composition provided by the supplier and the test report provided by a third-party testing organization. The expended materials can be checked for compliance with product substance content (or element content) of the relevant regulations. In addition, the product substance inspection system and product substance inspection method can also determine whether the content of the analyzed test report is consistent with the aggregate material composition, list the details for the non-conforming substance items, and provide alternative material composition for improvement suggestions. In this way, the alternative solutions can be quickly found, and effective risk management can be performed on the provided materials to ensure the safety of product materials. In addition, the product substance inspection system and the product substance inspection method can also be adjusted according to preference settings to automatically output improvement suggestions.

The method of the present invention, or a specific type or part thereof, can exist in the form of code. The code can be included in physical media, such as floppy disks, CDs, hard disks, or any other machine-readable (such as computer-readable) storage media, or computer program products that are not limited to external forms. When the program code is loaded and executed by a machine, such as a computer, the machine becomes a device for implementing the present invention. The code can also be transmitted through some transmission media, such as wire or cable, optical fiber, or any transmission type. When the program code is received, loaded and executed by a machine, such as a computer, the machine becomes a device for participating in the present invention. When implemented in a general-purpose processing unit, the program code combined with the processing unit provides a unique device that operates similar to the application of specific logic circuits.

Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such a feature may be combined with one or more other features of the other implementations as may be desired and advantageous for any given or particular application.

What is claimed is:

1. A product substance inspection system, comprising:
an expansion module, configured to expend regulatory information into a plurality of substance thresholds;
an inspection module, configured to obtain material composition corresponding to a constituent from aggregate material composition stored in a database, and compare each element in the material composition with the corresponding substance threshold; and
a preference-setting module, wherein when the inspection module determines that the value of a specific element in the material composition exceeds the corresponding substance threshold, the preference-setting module replaces the constituent to which the specific element belongs according to preference settings.

2. The product substance inspection system of claim 1, wherein the aggregate material composition comprises all the constituent of an end product, each of the constituent has a material composition, and the material composition comprises the elements in the constituent.

3. The product substance inspection system of claim 1, wherein when the inspection module determines that the amount of a specific element in the material composition exceeds the corresponding substance threshold, the preference-setting module selects an alternative constituent to replace the constituent according to the preference settings.

4. The product substance inspection system of claim 3, further comprising:
an improvement-suggestion module, configured to obtain a plurality of weights in the preference settings, calculate a weighted score for each of a plurality of candidate alternative constituents based on the weights, and select the largest one of the weighted scores for use as the alternative constituent.

5. The product substance inspection system of claim 1, further comprising:
an identification module, configured to receive a test report of an end product from a third-party server or a manufacturer's server, parse the test report into the aggregate material composition, and store the aggregate material composition in the database.

6. A product substance inspection method, comprising:
expending regulatory information into a plurality of substance thresholds by an expansion module;
obtaining the material composition of a constituent from the aggregate material composition stored in a database by an inspection module; and
comparing each element in the material composition with the corresponding substance threshold by a preference-setting module;
wherein, when the inspection module determines that the value of a specific element in the material composition exceeds the corresponding substance threshold, a preference-setting module replaces the constituent to which the specific element belongs according to the preference settings.

7. The product substance inspection method of claim 6, wherein the aggregate material composition comprises all the constituent of an end product, each of the constituent has a material composition, and the material composition comprises the elements in the constituent.

8. The product substance inspection method of claim 6, further comprising:
when the inspection module determines that the amount of a specific element in the material composition exceeds the corresponding substance threshold, the preference-setting module selects an alternative constituent to replace the constituent according to the preference settings.

9. The product substance inspection method of claim 8, further comprising:
obtaining a plurality of weights in the preference settings, calculating a weighted score for each of a plurality of candidate alternative constituents based on the weights, and using the improvement-suggestion module to select the largest one of the weighted scores for use as the alternative constituent.

10. The product substance inspection method of claim 6, further comprising:
receiving a test report of an end product from a third-party server or a manufacturer's server, parsing the test report into the aggregate material composition, and using an identification module to store the aggregate material composition in the database.
